(19) United States
(12) Patent Application Publication
(10) Pub. No.: US 2020/0170952 A1
(43) Pub. Date: Jun. 4, 2020

(54) Bubblyte

(71) Applicants: STC.UNM, Albuquerque, NM (US); Swinburne University of Technology, Hawthorn, Victoria (AU)

(72) Inventors: Heather Canavan, Albuquerque, NM (US); Phuong Anh Nguyen, Albuquerque, NM (US); Sally McArthur, Hawthorne (AU); Darnell Leon Cuylear, Albuquerque, NM (US)

(73) Assignees: STC.UNM, Albuquerque, NM (US); Swinburne University of Technology, Hawthorn, Victoria (AU)

(21) Appl. No.: 16/618,011

(22) PCT Filed: May 31, 2018
(86) PCT No.: PCT/US2018/035506

§ 371 Date: Nov. 27, 2019

Related U.S. Application Data
(60) Provisional application No. 62/513,952, filed on Jun. 1, 2017.

(51) Int. Cl. A61K 9/16; A61K 9/50; A61K 33/00; A61K 33/14

(52) U.S. Cl. CPC A61K 9/1641; A61K 33/14; A61K 33/00; A61K 9/5036

(57) ABSTRACT

A preparation comprised of one or more pellets having an outer shell and core. The outer shell is made of dissolvable material and said core containing poly (ethylene glycol).

### RELATED APPLICATIONS

This application claims the benefit of U.S. Provisional Application No. 62/513,952 filed on Jun. 1, 2017 and herein incorporated by reference.

### STATEMENT REGARDING FEDERALLY SPONSORED RESEARCH & DEVELOPMENT

This invention was made with government support under GM008751 awarded by the National Institutes of Health (NIH). The government has certain rights in the invention.

### BACKGROUND OF THE INVENTION

Colorectal cancer (CRC) is the second leading cause of cancer death in the United States. It is now the 4th in the top 10 list of the most common types of cancer in the United States with an estimated 100,000 new cases per year. The most reliable screening method of CRCs is colonoscopy, a procedure that allows for the visualization and removal of benign polyps that cause 75 to 80% of CRCs. A complete colonoscopy screening can reduce CRC incidents by 83%, and CRC mortality rates by 89%.

Although screening for CRC has been shown to reduce the incidence rate and mortality rate of the disease and is recommended by the CDC, only 58% of adults from 50-75 are up-to-date with their CRC screening (as of 2013). This disparity is often attributed to the anxiety surrounding the procedure, as well as the discomfort associated with the procedure.

In fact, a large proportion of patients and physicians who are interviewed on the reasons why patients do not perform colonoscopy report that patients are afraid of the preparation required for the procedure, Approximately 34% of all patients who do obtain the procedure reported that they experience moderate to significant discomfort in association with the preparation.

The standard colonoscopy preparation technique is to drink 4 L (˜1 gallon) of poly(ethylene glycol) electrolyte lavage solution (PEG-ELS) (see Table 1) the night prior to the procedure. The mixture is frequently described as foul tasting, with side effects of nausea, vomiting, abdominal bloating, abdominal pain, rectal irritation, etc. Frequently, patients cannot complete their required dose due to these side effects, which leads to incomplete bowel cleansing, poorly visualized colons that can result in insufficient removal of polyps. In some cases, this leads to canceled colonoscopies which must be rescheduled, and the entire process repeated, Table 1 includes bowel preparation products currently on the market, including the four most commonly used preparations: 1) PEG-ELS, 2) SF-PED-ELS (sulfate-free, for patients with sulfa allergy), 3) PEG-ELS with ascorbic acid (vitamin C, for electrolyte balance), and 4) low-volume PEG-3350-SD (½ the water necessary for prep),

TABLE 1

| | | | Low-volume | |
|---|---|---|---|---|
| | | | PEG-ELS with | Low-volume |
| | PEG-ELS | SF-PEG-ELS | ascorbic acid | PEG-3350-SD |
| Brand name | GoLYTELY | NuLYTELY; Trilyte | Moviprep | Miralax |
| Company (location) | Braintree Laboratories | Braintree Laboratories | Salix Pharmaceuticals | Merck (Boston, MA) |
| | (Braintree, Mass) | | (Raleigh, NC) | |
| Composition | PEG, sodium sulfate, | PEG, sodium | PEG-3350, sodium | PEG-3350 |
| | sodium, bicarbonate, | bicarbonate, | sulfate, sodium | |
| | sodium chloride, | sodium chloride, | chloride, ascorbic | |
| | potassium chloride | potassium chloride | acid | |
| Purgative volume/amount; | 4 L; none | 4 L; none | 2 L; | 238 g PEG-3350 in |
| recommended minimum | | | 1 L clear liquid | 2 L SD; |
| additional fluid | | | | regimens vary |
| (per prescribing information) | | | | |
| for FDA approved products) | | | | |
| FDA approval | Yes | Yes | Yes | Yes |
| Average wholesale | 24.56 | 26.89 (NuLYTELY) | 81.17 | 10.08 |
| price, US$ | | 27.98 (Trilyte) | | |
| Dosing regiments | Split-dose: 2-3 L day | Split-dose: 2-3 L day | Split-dose: 1 L day | Split-dose: 1 L day |
| | before and 1-2 L day | before and 1-2 L day | before and 1 L day | before and 1 L day |
| | of procedure | of procedure | of procedure | of procedure Single |
| | Single dose: 4 L day | Single dose: | Single dose: | dose: 2 L day before |
| | before | 4 L day before | 2 L day before | |
| Specific comments | Criterion standard; | More palatable | Avoid in patients with | Not balanced ELS; |
| | least palatable | than PEG-ELS | glucose-6-phosphate | unclear whether |
| | preparation | | dehydrogenase | electrolyte shifts |
| | | | deficiency | may occur |

### BRIEF SUMMARY OF THE INVENTION

In one embodiment, the present invention provides a method, approach and solution that can transfer poly (ethylene glycol) or PEG in a solid form that then dissolves in the stomach to achieve its goals.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates an exemplary delivery system for an embodiment of the present invention.

FIG. 2 illustrates drug release for an embodiment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

In one embodiment, the present invention provides a method, approach and solution that can transfer poly(ethylene glycol) or PEG in a solid form that then dissolves in the stomach to achieve its goals. In a preferred embodiment, the present invention provides a hydrogel pellet as the solid form and is configured to contain PEG-3350. Using a pellet form makes it easier to ingest, without the side effects associated with the unpalatable nature of PEG-3350 in liquid form.

Each individual pellet may be adapted to provide a unit or standard dosage. In a preferred embodiment, the unit dosage contains 17 g of PEG 3350, a dosage similar to those found in Miralax, a product used to aid in chronic constipation.

For use in bowel preparation, a standard dose may consist of a total of around 13 pellets to be ingested by the patient throughout the day with their drink of choice. This dose may be adjusted for variations in patient height and weight, unlike the currently available products. Each pellet may also contain sulfate, 0.518 g of sodium bicarbonate, 0.451 g of sodium chloride, 0.228 g of potassium chloride) to prevent symptoms of dehydration in association with bowel preparation for colonoscopies.

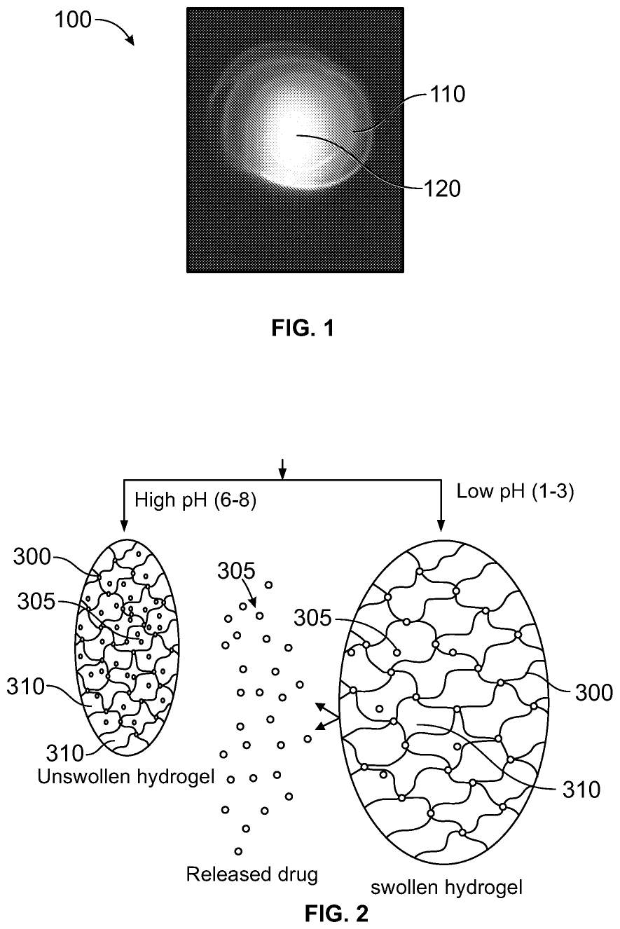

As shown in FIG. 1, a preferred embodiment of the present invention uses a hydrogel pellet as the solid form 100. Hydrogel pellet 100 has a pH-sensitive shell 110 defining an inner core 120. PEG-3350 200 and other compounds may be located in core 120.

The outer shell 110 of the hydrogel may be composed of chitosan and alginate, two materials that have been FDA approved in the past due to their biocompatible properties. As shown in FIG. 2, chitosan and alginate may be cross-linked to form a mesh 300 that encapsulates the PEG-3350 305.

In the acidic conditions of the stomach, the gel forming mesh 300 will swell. Swelling causes mesh 300 to widen and PEG-3350 305 will be released for bowel cleansing.

Chitosan and alginate have been cross-linked in a variety of methods to produce a pH-responsive hydrogel for targeted intestinal drug delivery. This selective pH response permits selective release of the compound. For example, different commonly used drinks (e.g., sodas, carbonated drinks, juices, etc.) and their pH may also be used as triggers. Thus, giving patients freedom to choose a drink of their choice for bowel preparation.

Pellet size may also be optimized to ensure hydrogels are approximately pill-sized, no larger than the typical over the counter drug found in pharmacies throughout the United States.

In yet another embodiment, the present invention provides a hydrogel that is pH responsive and will release PEG-3350 at stomach pH levels.

In yet other embodiments, the present invention provides pellet sizes that are easy to ingest-no larger than the size of over the counter drugs found in pharmacies throughout the U.S. The embodiment may also contain PEG-3350 with electrolytes that are ideal for bowel cleansing.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A preparation comprising: a pellet having an outer shell and core, said outer shell made of a dissolvable material and said core containing PEG-3350.

2. The preparation of claim 1 wherein said dissolvable material is a hydrogel.

3. The preparation of claim 2 wherein said hydrogel is composed of chitosan and alginate.

4. The preparation of claim 2 wherein said hydrogel is composed of chitosan and alginate which are cross-linked to encapsulate the PEG-3350.

5. The preparation of claim 1 wherein the preparation is pH responsive.

6. The preparation of claim 1 wherein the preparation is pH responsive at a pH of 1-3.

7. The preparation of claim 4 further including a plurality of pellets, each of said pellet contains 17 g of PEG 3350, sulfate, 0.518 g of sodium bicarbonate, 0.451 g of sodium chloride, and 0.228 g of potassium chloride.

8. The preparation of claim 4 wherein the preparation is configured to form a mesh, said mesh swells at a pH of 1-3 to release said PEG-3350 from said core.

9. The preparation of claim 8 wherein the preparation releases said PEG-3350 as the hydrogel swells and the mesh widens causing said PEG-3350 to be released from said core.

10. A preparation comprising: a pellet having an outer shell and core, said outer shell made of a dissolvable material and said core containing poly(ethylene glycol).

11. The preparation of claim 10 wherein said dissolvable material is a hydrogel.

12. The preparation of claim 11 wherein said outer shell of said hydrogel is composed of chitosan and alginate.

13. The preparation of claim 11 wherein said outer shell of the hydrogel is composed of chitosan and alginate which are cross-linked to encapsulate said poly(ethylene glycol).

14. The preparation of claim 10 wherein the preparation is pH responsive.

15. The preparation of claim 10 wherein the preparation is pH responsive at a pH of 1-3.

16. The preparation of claim 13 further including a plurality of pellets, each of said pellet contains 17 g of poly(ethylene glycol), sulfate, 0.518 g of sodium bicarbonate, 0.451 g of sodium chloride, and 0.228 g of potassium chloride.

17. The preparation of claim 13 wherein the preparation is configured to swell at a pH of 1-3 to release said poly(ethylene glycol) from said core.

18. The preparation of claim 17 wherein the preparation releases said poly(ethylene glycol) as the hydrogel swells and the mesh widens causing said poly(ethylene glycol) to be released from said core.

19. A method of bowel preparation for colonoscopies comprising the steps of: administering an effective amount of a preparation, said preparation comprising a pellet having an outer shell and core, said outer shell made of a dissolvable material and said core containing poly(ethylene glycol).

20. The method of claim 19 wherein said dissolvable material is a hydrogel and said preparation releases said poly(ethylene glycol) as said hydrogel swells and a mesh formed by said hydrogel widens causing said poly(ethylene glycol) to be released from said core.

21. The method of claim 20 further including the step of administering an effective amount of a liquid that triggers the swelling of the mesh formed by said hydrogel.

22. The method of claim 20 further including the step of administering an effective amount of a liquid having a pH that triggers the swelling of the mesh formed by said hydrogel.
